(19) United States
(12) Patent Application Publication
(10) Pub. No.: US 2015/0232457 A1
(43) Pub. Date: Aug. 20, 2015

(54) CRYSTALLINE FORMS OF AFATINIB DI-MALEATE

(71) Applicants: Anja Bruns, Neu-Ulm (DE); Simone Eichner, Neu-Ulm (DE); Frank Lehmann, Ulm (DE); Wolfgang Albrecht, Ulm (DE); Annemarie Maier, Biberach (DE)

(72) Inventors: Anja Bruns, Neu-Ulm (DE); Simone Eichner, Neu-Ulm (DE); Frank Lehmann, Ulm (DE); Wolfgang Albrecht, Ulm (DE); Annemarie Maier, Biberach (DE)

(73) Assignee: RATIOPHARM GMBH, Ulm (DE)

(21) Appl. No.: 14/349,217

(22) PCT Filed: Apr. 25, 2012
(86) PCT No.: PCT/US12/34903

§ 371 Date: Sep. 18, 2014

Related U.S. Application Data
(60) Provisional application No. 61/544,108, filed on Oct. 6, 2011.
(60) Provisional application No. 61/590,431, filed on Jan. 25, 2012.

(51) Int. Cl. C07D 405/12

(52) U.S. Cl. CPC C07D 405/12

(57) ABSTRACT

Crystalline forms of Afatinib di-maleate are described in the present application and processes for their preparation. The present invention also includes pharmaceutical compositions of such crystalline forms of Afatinib di-maleate, methods of their preparation and the use thereof hi the treatment of a patient in need thereof. The present invention also describes preparing Afatinib free base and salts of Afatinib, other than Afatibin di-maleate, and solid forms thereof.

### CROSS REFERENCE TO RELATED APPLICATIONS

This patent application claims the benefit of U.S. Provisional Patent Application Nos. 61/544,108 filed Oct. 6, 2011, and 61/590,431, filed Jan. 25, 2012, the disclosure of which provisional applications are herein incorporated by reference.

### FIELD OF THE INVENTION

The present invention encompasses crystalline forms of Afatininb di-maleate.

### BACKGROUND OF THE INVENTION

The compound, (E)-4-Dimethylamino-but-2-enoic acid {4-(3-chloro-4-fluoro-phenylamino)-7-[(S)-(tetrahydro-furan-3-yl)oxy]-quinazolin-6-yl}-amide, known as Afatinib, having the following structure:

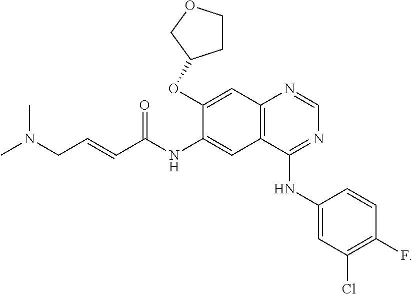

is an investigational orally administered irreversible inhibitor of both the epidermal growth factor receptor (EGFR) and human epidermal receptor 2 (HER2) tyrosine kinases.

Afatinib is under development for treatment of several solid tumors including non-small cell lung cancer (NSCLC), breast, head and neck cancer, and a variety of other cancers.

WO2002/50043 and WO2005/037824 (WO′824) describe Afatinib, a salt thereof and a crystalline form of the di-maleate salt.

The present invention relates to solid state forms of Afatinib di-maleate which possess different physical properties. The solid state form and the associated properties can be influenced by controlling the conditions under which Afatinib di-maleate is obtained in solid form.

Polymorphism, the occurrence of different crystal forms, is a property of some molecules and molecular complexes. A single molecule may give rise to a variety of polymorphs having distinct crystal structures and physical properties like melting point, thermal behaviors (e.g. measured by thermogravimetric analysis—“TGA”, or differential scanning calorimetry—“DSC”), X-ray powder diffraction (XRPD or powder XRD) pattern, infrared absorption fingerprint, and solid state nuclear magnetic resonance (NMR) spectrum. One or more of these techniques may be used to distinguish different polymorphic forms of a compound.

Discovering new polymorphic forms and solvates of a pharmaceutical product can provide materials having desirable processing properties, such as ease of handling, ease of processing, storage stability, ease of purification or as desirable intermediate crystal forms that facilitate conversion to other polymorphic forms. New polymorphic forms and solvates of a pharmaceutically useful compound or salts thereof can also provide an opportunity to improve the performance characteristics of a pharmaceutical product. It enlarges the repertoire of materials that a formulation scientist has available for formulation optimization, for example by providing a product with different properties, e.g., better processing or handling characteristics, improved dissolution profile, or improved shelf-life. For at least these reasons, there is a need for additional solid state forms of Afatinib di-maleate.

### SUMMARY OF THE INVENTION

The present invention provides crystalline forms of Afatinib di-maleate, processes for preparing them, and pharmaceutical compositions containing them.

The present invention also encompasses the use of any one of the crystalline forms of Afatinib di-maleate provided herein, for the preparation of Afatinib, other Afatinib salts, solid state forms thereof, and formulations thereof.

The present invention also encompasses the use of any one of the crystalline forms of Afatinib di-maleate disclosed herein for the preparation of a medicament, preferably for the treatment of cancer, particularly for the treatment of cancers mediated by epidermal growth factor receptor (EGFR) and human epidermal receptor 2 (HER2) tyrosine kinases, e.g., solid tumors including NSCLC, breast, head and neck cancer, and a variety of other cancers mediated by EGFR or HER2 tyrosine kinases.

The present invention further provides a pharmaceutical composition comprising any one of the Afatinib di-maleate crystalline forms of the present invention and at least one pharmaceutically acceptable excipient.

The present invention also provides a method of treating cancer, comprising administering a therapeutically effective amount of at least one of the Afatinib di-maleate crystalline forms of the present invention, or at least one of the above pharmaceutical compositions to a person suffering from cancer, particularly a person suffering from a cancer mediated by epideimal growth factor receptor (EGFR) and human epidermal receptor 2 (HER2) tyrosine kinases, e.g., solid tumors including but not limited to NSCLC, breast, head and neck cancer, and a variety of other cancers mediated by EGFR or HERZ tyrosine kinases.

### BRIEF DESCRIPTION OF THE DRAWINGS

FIG. 1 shows an X-ray powder diffractogram of Afatinib di-maleate Form C.

FIG. 2 shows a DSC thermogram of Afatinib di-maleate Form C.

FIG. 3 shows a ¹H-NMR spectrum of Afatinib di-maleate Form C.

FIG. 4 shows an X-ray powder diffractogram of Afatinib di-maleate Form D.

FIG. 5 shows a DSC thermogram of Afatinib di-maleate Form D.

FIG. 6 shows a 1H-NMR spectrum of Afatinib di-maleate Form D.

FIG. 7 shows an X-ray powder diffractogram of Afatinib dimaleate Form E.

FIG. 8 shows a Humidity-dependent weight increase of a sample of Afatinib di-maleate Form A.

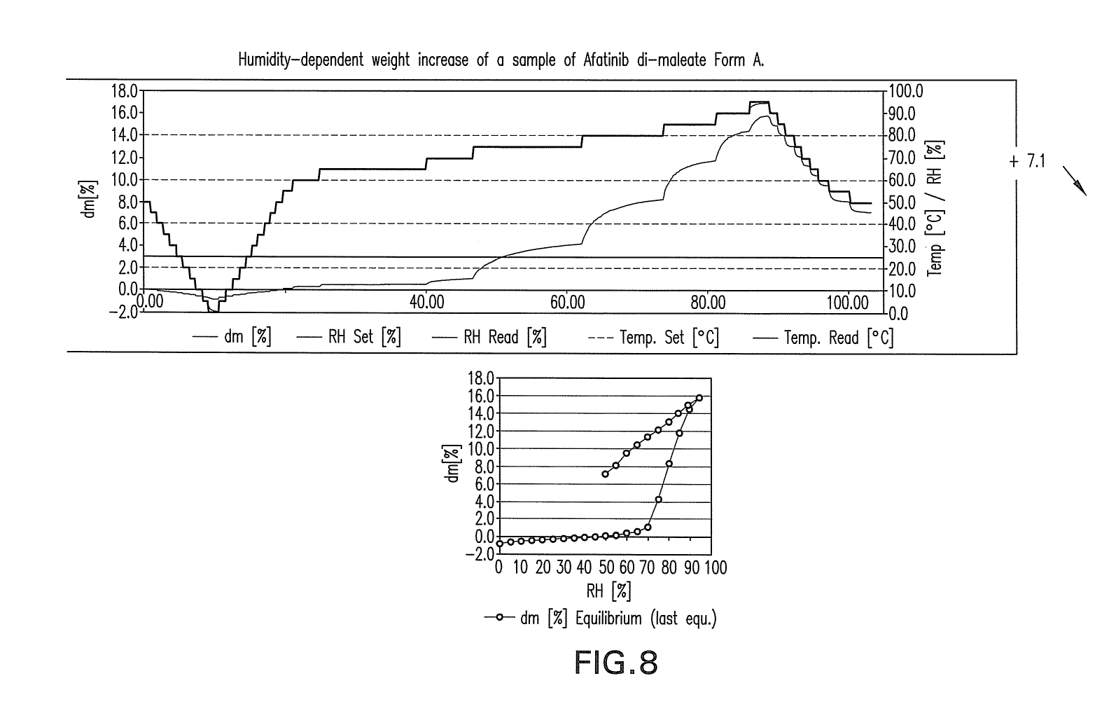

FIG. 9 shows an HPLC/UV chromatogram of analysis of Afatinib di-maleate Form A after storage for 4 weeks at 40° C./75% relative humidity.

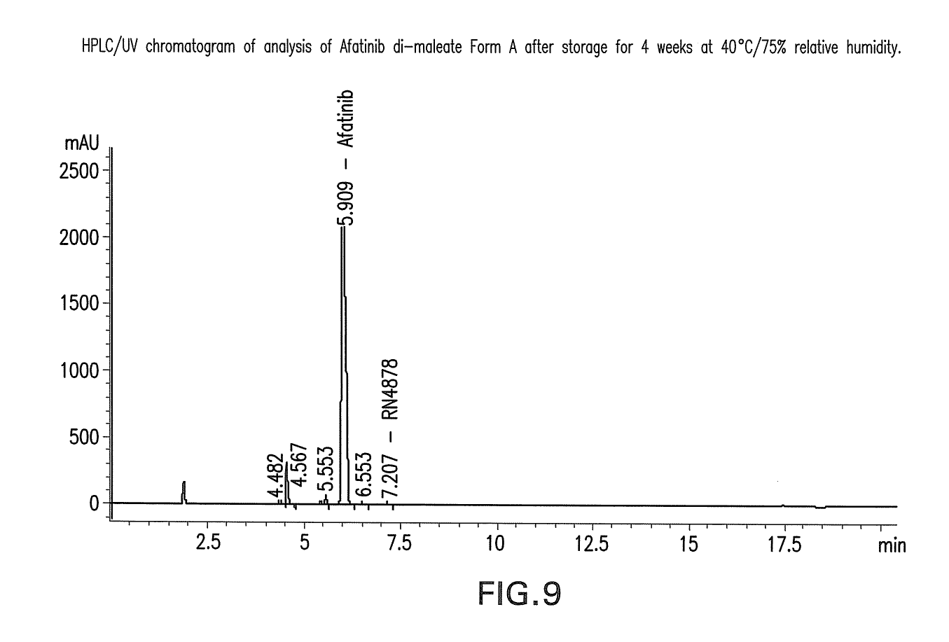

FIG. 10 shows a ¹H-NMR-spectrum of Afatinib di-maleate Form A after storage for 4 weeks at 40° C./75% relative humidity.

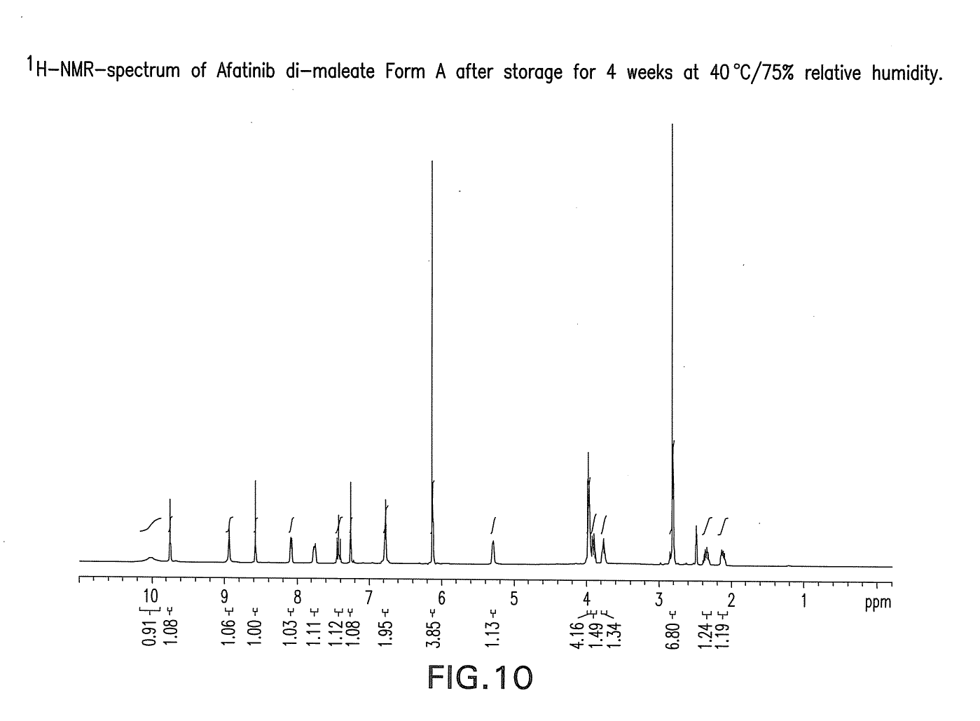

FIG. 11 shows an X-ray powder diffractogram of Afatinib di-maleate, Form A.

### DETAILED DESCRIPTION OF THE INVENTION

US 2005/0085495 (the US counterpart of WO′824) cites that “Afatinib di-maleate as suitable salt for pharmaceutical use as it exist in only one crystalline modification, which is moreover anhydrous and very stable. In addition, the described crystalline satisfies the physicochemical requirements”. Namely, this crystalline form has only limited hygroscopisity and is polymorphically stable.

Unlike what is written in US 2005/0085495, additional crystalline forms of Afatinib di-maleate were prepared as described herein, which posses better physicochemical features.

In some embodiments the crystalline forms of Afatinib di-maleate of the invention are substantially free of any other polymorphic forms, or substantially free of a specified polymorph of Afatinib di-maleate. In any embodiment of the present invention, by “substantially free” is meant that the forms of the present invention contain 20% (w/w) or less, 10% (w/w) or less, 5% (w/w) or less, 2% (w/w) or less, particularly 1% (w/w) or less, more particularly 0.5% (w/w) or less, and most particularly 0.2% (w/w) or less of any polymorphs or of a specified polymorph of Afatinib di-maleate. In other embodiments, the polymorphs of Afatinib di-maleate of the invention contain from 1% to 20% (w/w), from 5% to 20% (w/w), or from 5% to 10% (w/w) of any other polymorphs or of a specified polymorph of Afatinib di-maleate.

The present invention provides new crystalline forms of Afatinib di-maleate that have advantageous properties over other solid state forms of Afatinib di-maleate, selected from at least one of: chemical purity, flowability, solubility, dissolution rate, morphology or crystal habit, stability, such as thermal and mechanical stability to polymorphic conversion, stability to dehydration and/or storage stability, low content of residual solvent, a lower degree of hygroscopicity, flowability, and advantageous processing and handling characteristics such as compressibility, and bulk density.

A solid state form may be referred to herein as being characterized by data selected from two or more different data groupings, for example, by a powder XRD pattern having a group of specific peaks; or by a powder XRD pattern as shown in a figure depicting a diffractogram, or by “a combination thereof” (or “combinations thereof,” or “any combination thereof”), These expressions, e.g., “any combination thereof” contemplate that the skilled person may characterize a crystal form using any combination of the recited characteristic analytical data. For example, the skilled person may characterize a crystal form using a group of four or five characteristic powder XRD peaks, and supplement that characterization with one or more additional features observed in the powder X-ray diffractogram, e.g., an additional peak, a characteristic peak shape, a peak intensity, or even the absence of a peak at some position in the powder XRD pattern. Alternatively, the skilled person may in some instances characterize a crystal form using a group of four or five characteristic powder XRD peaks and supplement that characterization with one or more additional features observed using another analytical method, for example, using one or more characteristic peaks in a solid state NMR spectrum, or characteristics of the DSC thermogram of the crystal form that is being characterized.

A solid state may be referred to herein as being characterized by graphical data “as depicted in” a Figure. Such data include, for example, powder X-ray diffractograms and solid state NMR spectra. The skilled person will understand that such graphical representations of data may be subject to small variations, e.g., in peak relative intensities and peak positions due to factors such as variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Afatinib di-maleate referred to herein as being characterized by graphical data “as depicted in” a Figure will thus be understood to include any crystal forms of Afatinib di-maleate characterized with the graphical data having such small variations, as are well known to the skilled person, in comparison with the Figure.

The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount. When the solvent is present in stoichiometric amount, the hydrate may be referred to as monohydrate, di-hydrate, tri-hydrate etc. The solvent content can be measured, for example, by GC, ¹H-NMR, Karl-Fischer (KF) titration or by monitoring the weight increase during dynamic vapour sorption (DVS) test.

The term “anhydrous” as used herein, and unless stated otherwise, refers to crystalline Afatinib di-maleate which contains not more than 1% (w/w), preferably not more than 0.5% (w/w) of either water or organic solvents as measured by TGA.

As used herein, the term “isolated” in reference to any of Afatinib di-maleate polymorphs thereof of the present invention corresponds to Afatinib di-maleate polymorph that is physically separated from the reaction mixture, where it is formed.

The term “non-hygroscopic” as used herein, and unless stated otherwise, refers to crystalline Afatinib di-maleate uptaking/absorbing less than 0.2% (w/w) of atmospheric water to the crystalline Afatinib di-maleate in the below specified conditions, as measured by Karl-Fischer (KF) titration or by monitoring the weight increase during dynamic vapour sorption (DVS) test.

As used herein, unless stated otherwise, the XRPD measurements are taken using copper Kα radiation wavelength A, =1.5406 Å. For the avoidance of doubt, the XRPD values described herein were measured using the diffractometer and conditions described below.

A thing, e.g., a reaction mixture, may be characterized herein as being at, or allowed to come to “room temperature, often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, typically about 16 hours.

As used herein, the term “reduced pressure” refers to a pressure of about 10 mbar to about 50 mbar.

As used herein, the term Afatinib di-maleate form A refers to the crystalline form provided in WO2005/037824, disclosed in the table provided below.

| 2-Θ [°] | d-value [Å] | intensity I/Iₒ [%] |
|---|---|---|
| 4.91 | 18.0 | 47 |
| 6.42 | 13.8 | 33 |
| 7.47 | 11.8 | 27 |
| 8.13 | 10.9 | 30 |
| 10.37 | 8.53 | 30 |
| 11.69 | 7.56 | 2 |
| 12.91 | 6.85 | 20 |
| 13.46 | 6.58 | 3 |
| 13.66 | 6.48 | 2 |
| 14.94 | 5.93 | 11 |
| 16.58 | 5.34 | 12 |
| 17.19 | 5.15 | 36 |
| 17.87 | 4.96 | 5 |
| 19.43 | 4.57 | 38 |
| 19.91 | 4.46 | 100 |
| 20.84 | 4.26 | 13 |
| 21.33 | 4.16 | 21 |
| 21.58 | 4.12 | 12 |
| 22.25 | 3.992 | 15 |
| 22.94 | 3.873 | 32 |
| 23.67 | 3.756 | 9 |
| 24.82 | 3.584 | 7 |
| 25.56 | 3.482 | 37 |
| 26.71 | 3.335 | 9 |
| 27.46 | 3.245 | 4 |
| 28.37 | 3.143 | 8 |
| 30.71 | 2.909 | 3 |
| 29.31 | 3.045 | 4 |
| 29.57 | 3.019 | 4 |
| 31.32 | 2.854 | 10 |
| 32.31 | 2.769 | 4 |
| 33.10 | 2.705 | 5 |
| 33.90 | 2.643 | 1 |
| 34.84 | 2.573 | 2 |
| 35.71 | 2.512 | 1 |
| 36.38 | 2.467 | 1 |
| 36.96 | 2.430 | 1 |
| 37.99 | 2.367 | 2 |
| 39.94 | 2.255 | 5 |

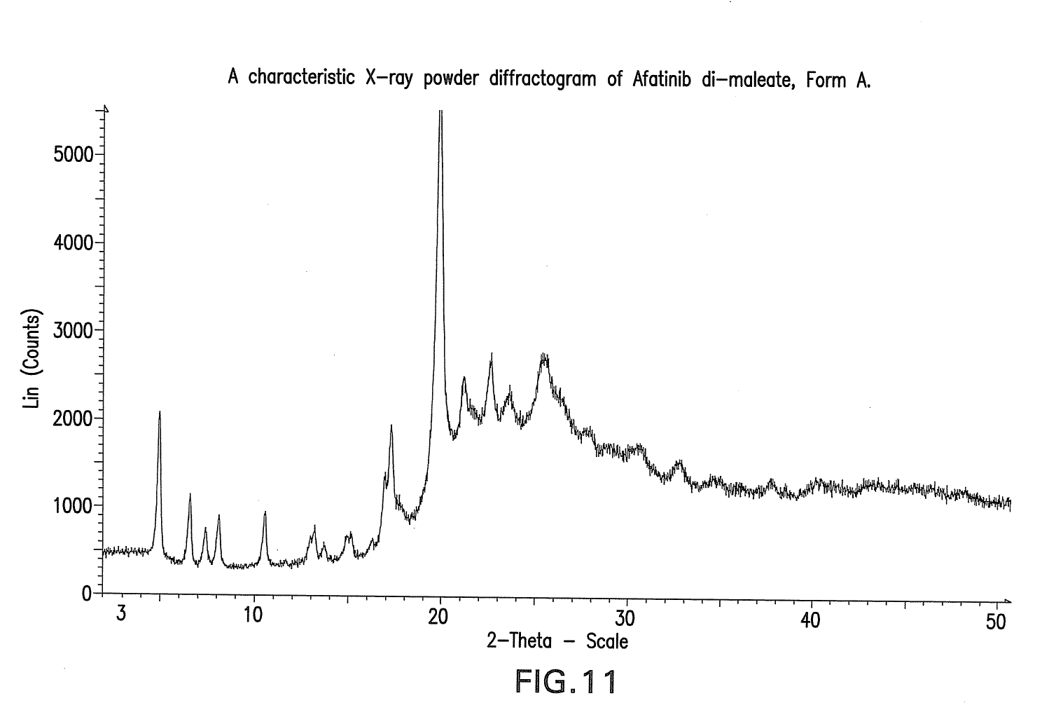

In particular, Afatinib di-maleate is characterized by an X-ray powder diffraction pattern substantially as depicted in FIG. 11 of the present application.

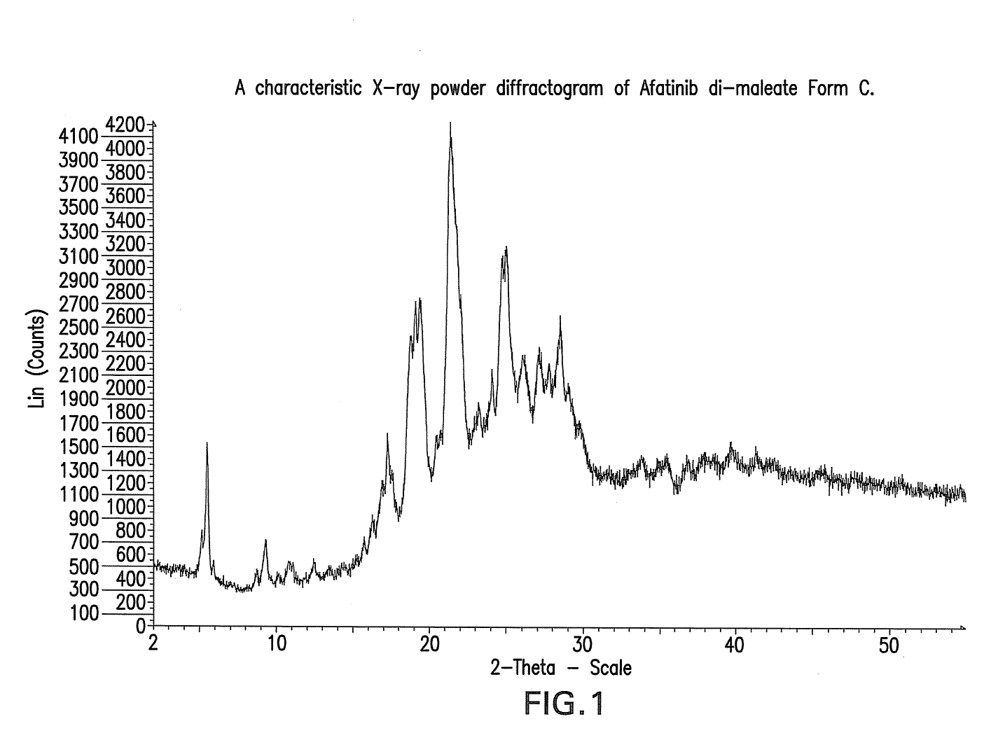

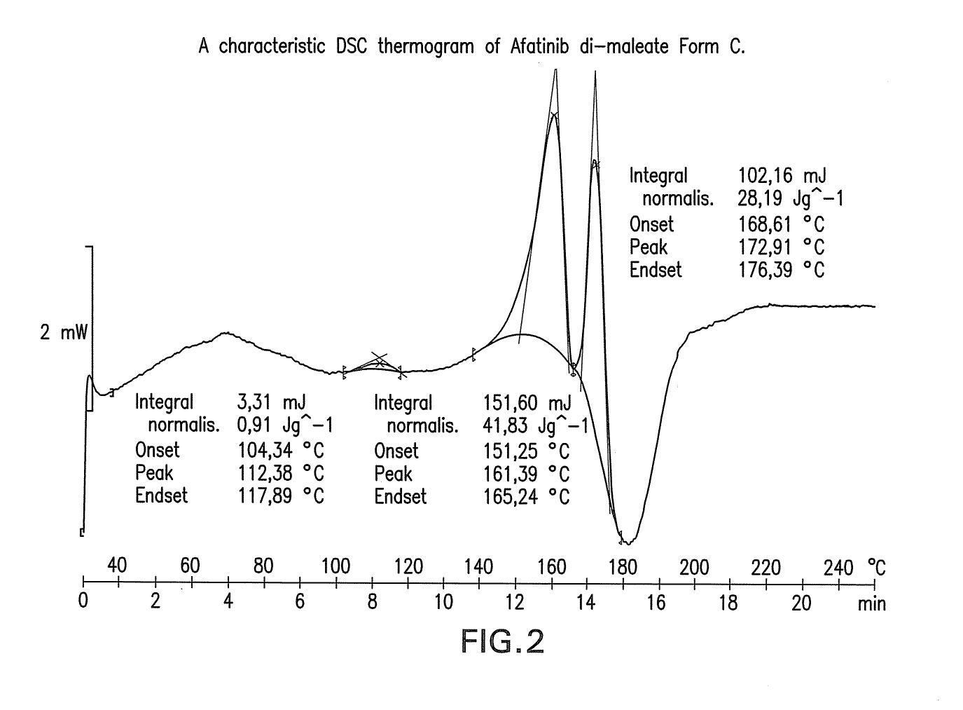

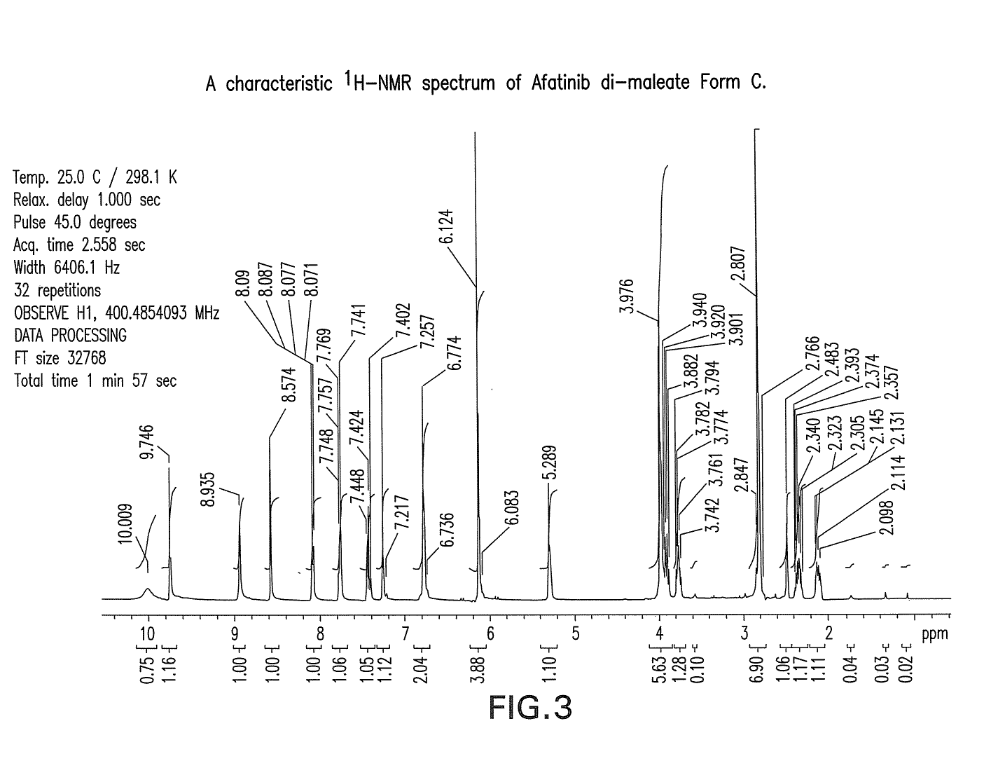

The present invention encompasses a crystalline form of Afatinib di-maleate, designated as Form C. Form C can be characterized by data selected from: an X-ray powder diffraction pattern having peaks at 5.5, 9.3, 18.8, 19.1 and 21.5 degrees two theta±0.2 degrees two theta; an X-ray powder diffraction pattern substantially as depicted in FIG. 1; and combinations thereof. Crystalline Form C of Afatinib di-maleate may be further characterized by additional analytical data selected from: an X-ray powder diffraction pattern having any one, two, three, four, five, six, seven or eight additional peaks selected from peaks at 5.1, 5.9, 8.7, 12.5, 15.7, 24.1, 26.2 and 28.6 degrees two theta±0.2 degrees two theta; a DSC thermogram substantially as depicted in FIG. 2; and a ¹H-NMR spectrum substantially as depicted in FIG. 3; and combinations thereof.

The above Afatinib di-maleate Form C may be anhydrous.

Form C of the present invention seems to have advantageous properties such as: chemical purity, flowability, solubility, dissolution rate, morphology or crystal habit, stability, such as thermal and mechanical stability to polymorphic conversion, stability to dehydration and/or storage stability, low content of residual solvent, a lower degree of hygroscopicity, flowability, and advantageous processing and handling characteristics such as compressibility, and bulk density.

According to some embodiments the crystalline Form C of Afatinib di-maleate of the invention are disclosed herein as being chemically stable under certain recited conditions, for example under conditions of 30° C./65% relative humidity for 4 weeks. By chemically stable is meant that the chemical purity of the Afatinib di-maleate when subjected to these conditions changes in an amount of less than about 1%; preferably less than about 0.8% w/w by HPLC, while the recited solid state form is maintained.

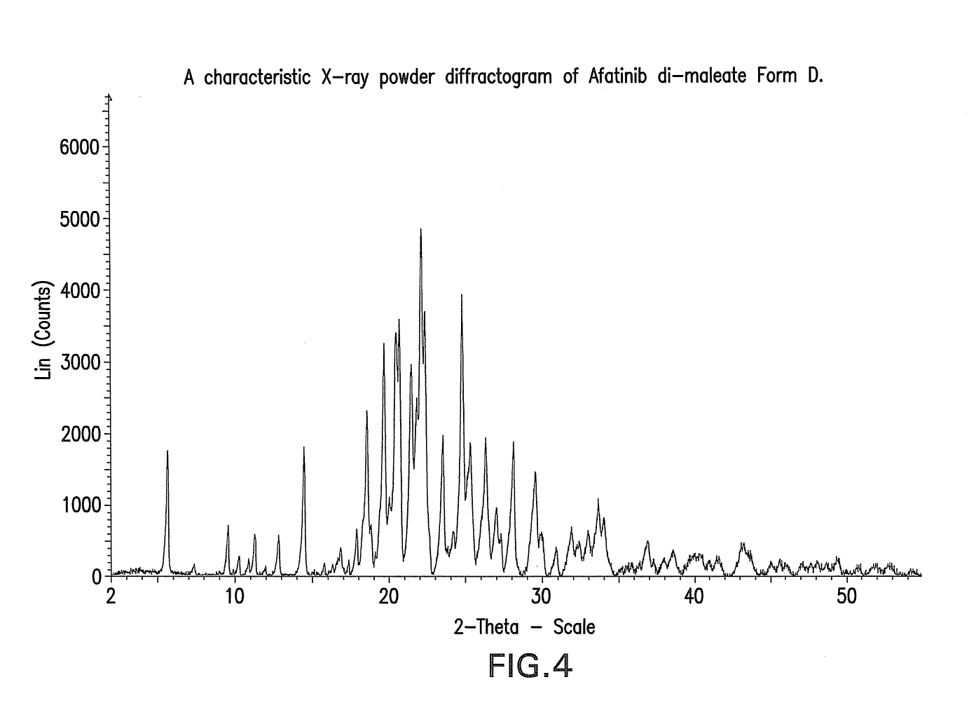

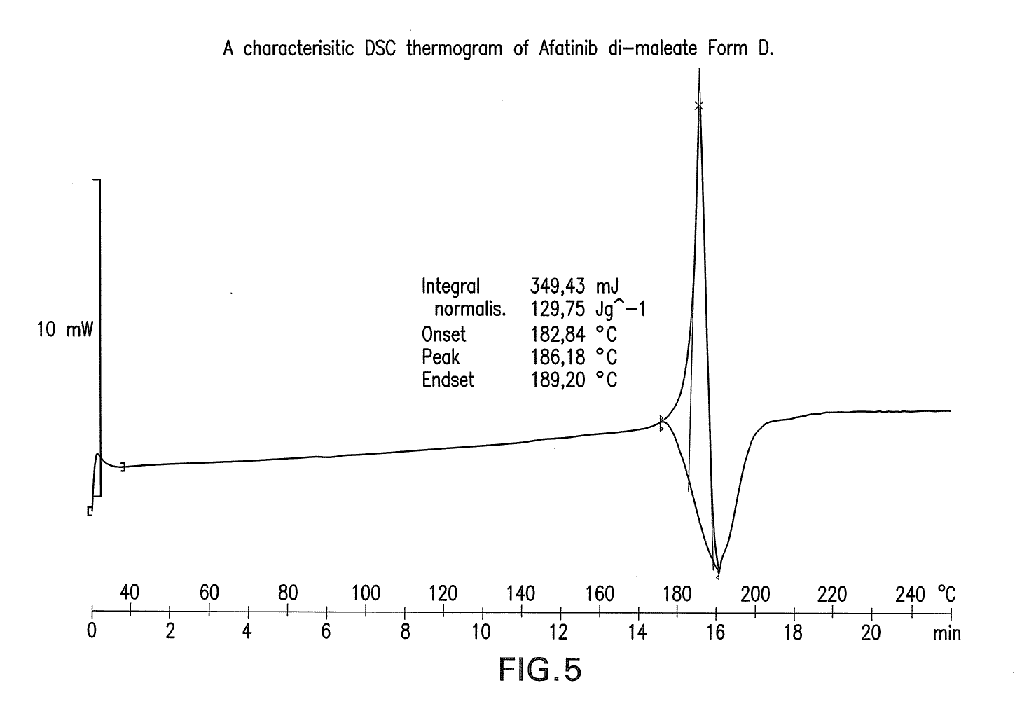

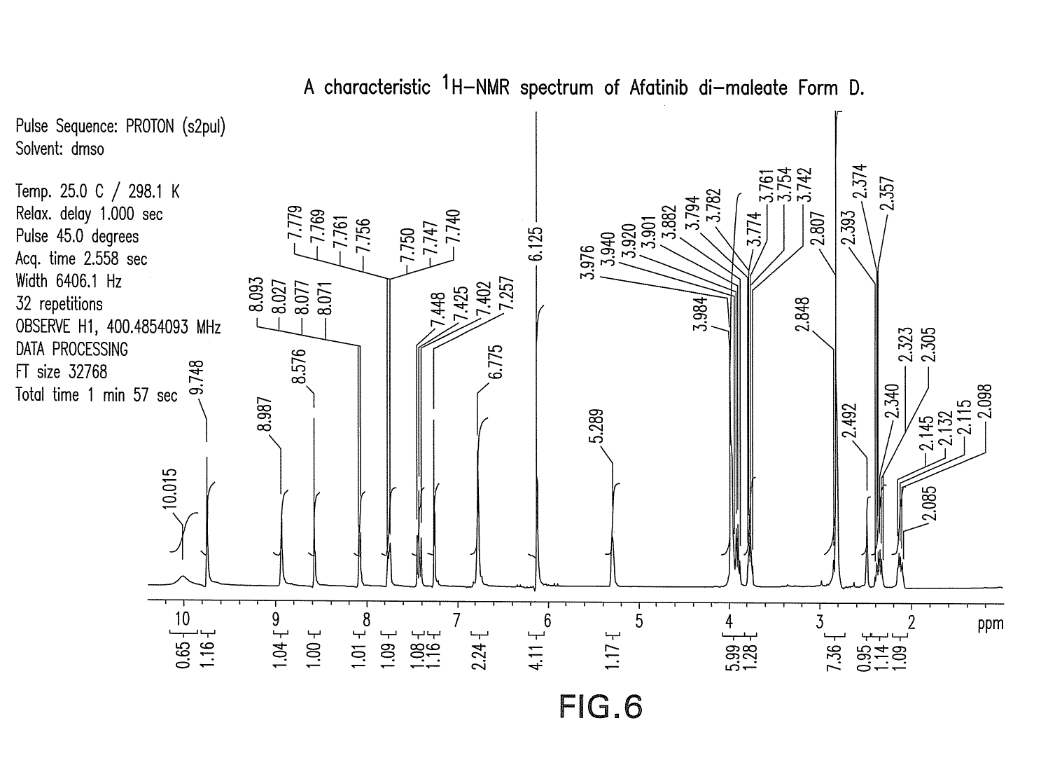

The present invention encompasses a crystalline form of Afatinib di-maleate, designated as Form D. Form D can be characterized by data selected from: an X-ray powder diffraction pattern having peaks at 5.6, 9.5, 22.1, 26.3 and 29.5 degrees two theta±0.2 degrees two theta; an X-ray powder diffraction pattern substantially as depicted in FIG. 4; and combinations thereof. Crystalline Form D of Afatinib di-maleate may be further characterized by additional analytical data selected from: an X-ray powder diffraction pattern having any one, two, three, four, five, six, seven, eight, nine or ten additional peaks selected from peaks at 11.2, 14.4, 18.5, 19.7, 20.5, 20.7, 22.3, 23.5, 24.8 and 28.1 degrees two theta±0.2 degrees two theta; a DSC thermogram substantially as depicted in FIG. 5; and a ¹H-NMR spectrum substantially as depicted in FIG. 6.

The above Afatinib di-maleate Form D may be anhydrous.

The present invention encompasses Afatinib di-maleate hydrate, for example, tri-hydrate.

The present invention encompasses a crystalline form of Afatinib di-maleate, designated as Form E. Form E can be characterized by data selected from: an X-ray powder diffraction pattern having peaks at 5.5, 11.4, 17.7, 22.3 and 25.5 degrees two theta±0.2 degrees two theta; an X-ray powder diffraction pattern substantially as depicted in

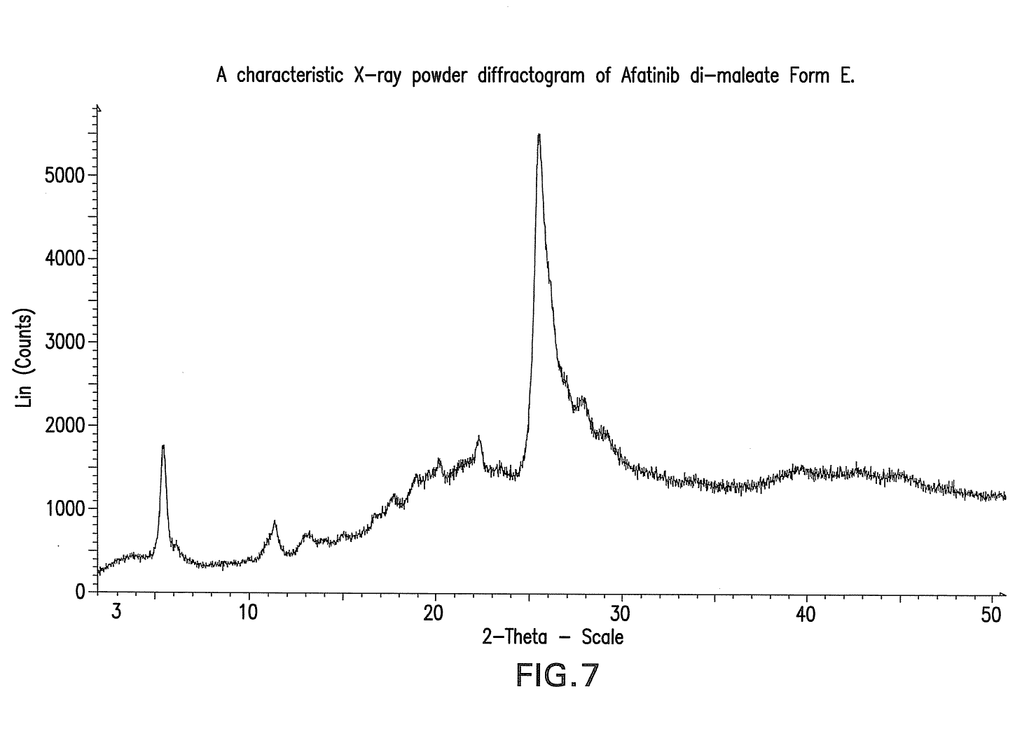

FIG. 7; and combinations thereof. Crystalline form of Afatinib di-maleate may be further characterized by additional analytical data selected from: an X-ray powder diffraction pattern having one, two, three, four or five additional peaks selected from peaks at 6.1, 13.1, 20.3, 28.0 and 29.1.

The above form E can be a hydrate form; particularly it can be a tri-hydrate form. The tri-hydrate form E can have a water content from about 5.9% to about 8.1%, for example of about 7% (w/w), or from about 2.5 mole equivalents to about 3.5 mole equivalents, for example of about, 3 mole equivalents of water per one mole equivalent of Afatinib di-maleate, as measured by Karl-Fischer (KF) titration or by monitoring the weight increase during dynamic vapour sorption (DVS) test.

Form E of the present invention seems to have advantageous properties such as: chemical purity, flowability, solubility, dissolution rate, morphology or crystal habit, stability, such as thermal and mechanical stability to polymorphic conversion, stability to dehydration and/or storage stability, low content of residual solvent, a lower degree of hygroscopicity, flowability, and advantageous processing and handling characteristics such as compressibility, and bulk density.

According to some embodiments the crystalline Form E of Afatinib di-maleate of the invention are disclosed herein as being polymorphically stable under certain recited conditions, for example under conditions of 40° C./75% relative humidity. By polymorphically stable is meant that under these conditions, less than 1% of the stable form converts to any other solid state form of Afatinib di-maleate. Further, under these conditions, form E has found to be not hygroscopic.

The above solid state forms of Afatinib di-maleate can be used to prepare 1) Afatinib free base and solid state forms thereof; 2) other Afatinib salts and solid state forms thereof; and 3) pharmaceutical formulations.

The present invention provides a process for preparing Afatinib free base, for example, by preparing any one of the solid state forms of the present invention; and basifying the said salt to obtain Afatinib free base. The process can further comprise converting the obtained Afatinib free base to any other salt of Afatinib and solid state forms thereof. The conversion can comprise, for example, reacting the obtained Afatinib free base with an appropriate acid to obtain the corresponding acid addition salt.

Alternatively, the conversion can be done by salt switching, i.e., reacting Afatinib di-maleate, with an acid having a pKₐ which is lower than the pKₐ of the acid of maleic acid.

The present invention further encompasses 1) a pharmaceutical composition comprising any one of Afatinib di-maleate crystalline forms, as described above, and at least one pharmaceutically acceptable excipient; and 2) the use of any one or combination of the above-described crystalline forms of Afatinib di-maleate, in the manufacture of a pharmaceutical composition, and 3) a method of treating a solid tumor such as NSCLC, breast, head and neck cancer, and a variety of other cancers, comprising administration of an effective amount of a pharmaceutical composition comprising any one or more of the forms of Afatinib di-maleate described herein.

The pharmaceutical composition can be useful for the treatment of solid tumors including NSCLC, breast, head and neck cancer, and a variety of other cancers. The present invention also provides crystalline forms of Afatinib di-maleate as described above for use as a medicament, preferably for the treatment of cancer, in particular, solid tumors including NSCLC, breast, head and neck cancer, and a variety of other cancers.

Having thus described the invention with reference to particular preferred embodiments and illustrative examples, those in the art can appreciate modifications to the invention as described and illustrated that do not depart from the spirit and scope of the invention as disclosed in the specification. The Examples are set forth to aid in understanding the invention but are not intended to, and should not be construed to limit its scope in any way.

### X-Ray Powder Diffraction (“XRPD”) Method:

Samples were analyzed on a D8 Advance X-ray powder diffractometer (Bruker-AXS, Karlsruhe, Germany). The sample holder was rotated in a plane parallel to its surface at 20 rpm during the measurement. Further conditions for the measurements are summarized below. The raw data were analyzed with the program EVA (Bruker-AXS, Germany).

| | standard measurement |
|---|---|
| radiation | Cu K_{α}(λ = \tilde{1}5\tilde{4}6 Å) |
| Source | 38 kV/40 mA |
| detector | Vantec |
| detector slit | variable |
| divergence slit | v6 |
| antiscattering slit | v6 |
| 2θ range/° | 2 ≦ 2θ ≦ 55 |
| step size/° | 0.017 |

### Differential Scanning Calorimetry:

Mettler Toledo Model DSC 822; heating range for the samples 30 to 250 deg C; heating rate=10 deg C/min; purge gas=nitrogen 50 ml/min; 40 micron aluminum crucible.

### ¹H-NMR Spectroscopy:

Instrument: Varian Mercury 400 plus NMR Spectrometer, Oxford AS, 400 MHz

### HPLC/UV

### Column: Phenomenex Kinetex 2.6μ C18 100A, 150*4.6 mm

### Oven: 40° C.

### λ: 260/4 nm Ref 550/100 nm

### Inj Vol.: 1 μl

### Eluent:

A: acetonitrile

B: 0.2% formic acid+0.1% HFBA pH 2.

Gradient:

| Time [min] | solvent B [%] | flow [ml/min] |
|---|---|---|
| 0 | 70 | 0.7 |
| 6 | 50 | 0.7 |
| 8 | 50 | 0.7 |
| 14 | 20 | 0.7 |
| 15 | 20 | 0.7 |
| 15.5 | 70 | 0.7 |
| 20.5 | Stop | |

### Karl-Fischer Titration

Karl-Fischer titration was carried out using Apura®-Testicit from Merck (HX908240). The instructions of the manual were followed and each sample was analyzed in triplicate.

### EXAMPLES

### Preparation of the Afatinib Base Starting Material

Afatinib base was prepared according to WO2005/037824 example 2.

5.6 litres of 30% hydrochloric acid (53.17 mol) are added to 4.4 liters of water. Then 4.28 kg of 95% (dimethylamino)-acetaldehyde-diethyl-acetal (26.59 mol) are added dropwise within 20 minutes at 30.degree. C. The reaction solution is stirred for 8 hours at 35.degree. C. stirred, cooled to 5.degree. C. and stored under argon. This solution is referred to as solution B.

4.55 kg (68.06 mol) of potassium hydroxide are dissolved in 23.5 liters of water and cooled to -5.degree. C. This solution is referred to as solution C.

5.88 kg (10.63 mol) of diethyl ((4-(3-chloro-4-fluoro-phenylamino)--7-(tetrahydrofuran-3-yloxy)-quinazoline-6-ylcarbamoyl)-methyl)-phosphonate and 0.45 kg of lithium chloride (10.63 mol) are placed in 23.5 liters of tetrahydrofuran and cooled to -7.degree. C. The cold solution C is added within 10 minutes. Then solution B is added at −7.degree. C. within 1 hour. After stirring for a further hour at −5.degree. C. the reaction mixture is heated to 30.degree. C. and combined with 15 litres of water. After cooling to 3.degree. C. the suspension is suction filtered, the precipitate is washed with water and dried. Yield: 5.21 kg of crude product, 100%, water content: 6.7%

The crystallisation of the crude product is carried out with butyl acetate/methylcyclohexane Yield: 78% purity HPLC 99.4FI %, water content 5.4%.

### Example 1

### Preparation of Afatinib Di-Maleate Form C

Afatinib free base (3 g) was dissolved in tetrahydrofuran (THF) (7.6 mL) and stirred at room temperature until a clear solution was obtained. While stirring the clear solution, a solution of maleic acid (1.48 g) in THF (7.6 mL) was added dropwise at room temperature. After completion of this addition, a suspension containing a sticky solid was obtained. THF (60 mL) was added to the suspension and this mixture was stirred at room temperature overnight. A solid precipitate Ruined and was collected by filtration and washed with THF (30 mL) to yield a yellowish solid. The product was dried at 40° C. and 20 mbar (yield: 4.28 g, 96.5%). XRPD peak data for the product is provided in the Table below.

| Angle (2Θ) | D value (Å) | Intensity % |
|---|---|---|
| 5.165 | 17.09611 | 18.9 |
| 5.527 | 15.97735 | 36.2 |
| 5.926 | 14.903 | 12.9 |
| 8.735 | 10.11492 | 11.3 |
| 9.318 | 9.48368 | 17.2 |
| 10.12 | 8.73385 | 10.5 |
| 10.847 | 8.14996 | 12.9 |
| 11.075 | 7.98276 | 12.7 |
| 12.484 | 7.08486 | 12.8 |
| 13.488 | 6.55952 | 12.1 |
| 15.726 | 5.6305 | 17.9 |
| 16.285 | 5.43851 | 22.1 |
| 16.934 | 5.23157 | 29 |
| 17.29 | 5.12473 | 38.3 |
| 17.546 | 5.05056 | 31 |
| 18.819 | 4.71161 | 57.7 |
| 19.131 | 4.63556 | 64.5 |
| 19.425 | 4.56588 | 65.1 |
| 20.50 | 4.32888 | 38 |
| 21.483 | 4.13305 | 100 |
| 21.835 | 4.06722 | 79.3 |
| 22.113 | 4.01664 | 64.2 |
| 23.034 | 3.85807 | 40.5 |
| 23.291 | 3.81608 | 43.3 |
| 24.135 | 3.68456 | 51.1 |
| 24.802 | 3.58686 | 73.4 |
| 25.06 | 3.55058 | 75.3 |
| 26.156 | 3.40419 | 54 |
| 27.148 | 3.28209 | 54.1 |
| 27.831 | 3.20305 | 52.3 |
| 28.564 | 3.12244 | 61.8 |
| 29.082 | 3.06801 | 48.3 |
| 29.769 | 2.99873 | 40.9 |
| 33.878 | 2.64391 | 33.4 |
| 35.441 | 2.53074 | 33.7 |
| 36.839 | 2.43786 | 33.0 |
| 39.675 | 2.26988 | 37.0 |
| 41.327 | 2.1829 | 36.0 |

### Example 2

### Preparation of Afatinib Di-Maleate Form D

A suspension of afatinib free base (1 g) in 96% EtOH (14.5 mL) was heated to 70° C. until a yellowish clear solution was obtained. While stirring this solution at 70° C., a solution of maleic acid (0.49 g) in EtOH (6 mL) was added dropwise. After completion of this addition, the reaction mixture was stirred for 15 minutes at 70° C. and then cooled slowly to ambient temperature. The cooled mixture was stirred at room temperature overnight, then cooled to 0° C. and stirred for another hour. A solid precipitate formed and was collected by filtration, washed with ethanol (6 mL) and dried at 40° C. and 20 mbar (yield: 1.26 g, 85.3%). XRPD peak data for the product is provided in the Table below.

| Angle (2Θ) | d value (Å) | Intensity % |
|---|---|---|
| 5.598 | 15.77302 | 36.1 |
| 7.303 | 12.0942 | 3.3 |
| 9.492 | 9.31022 | 14.4 |
| 10.203 | 8.66292 | 5.4 |
| 10.829 | 8.1634 | 4.9 |
| 11.233 | 7.87046 | 11.9 |
| 11.909 | 7.42538 | 2.3 |
| 12.77 | 6.92667 | 11.7 |
| 14.429 | 6.13357 | 37.2 |
| 15.739 | 5.62609 | 3.6 |
| 16.311 | 5.43 | 3.2 |
| 16.822 | 5.26622 | 8.1 |
| 17.333 | 5.11218 | 3.6 |
| 17.86 | 4.96236 | 13.5 |
| 18.541 | 4.78162 | 47.7 |
| 18.842 | 4.70591 | 13.6 |
| 19.67 | 4.50976 | 67 |
| 20.012 | 4.43344 | 22.6 |
| 20.478 | 4.33349 | 70.1 |
| 20.683 | 4.29103 | 74 |
| 21.462 | 4.13697 | 60.9 |
| 21.817 | 4.07043 | 51.3 |
| 22.131 | 4.01335 | 100 |
| 22.325 | 3.9789 | 72.3 |
| 23.5 | 3.78261 | 40.6 |
| 24.169 | 3.67937 | 12.8 |
| 24.777 | 3.59054 | 81.1 |
| 25.288 | 3.51905 | 38.5 |
| 26.298 | 3.38616 | 39.9 |
| 26.985 | 3.30151 | 19.6 |
| 27.262 | 3.26862 | 10.5 |
| 28.087 | 3.17441 | 36.6 |
| 29.247 | 3.05115 | 14.1 |
| 29.524 | 3.02309 | 30.1 |
| 29.914 | 2.98455 | 12.1 |
| 30.902 | 2.89137 | 8.2 |
| 31.88 | 2.80483 | 14.1 |
| 32.397 | 2.76127 | 10 |
| 32.976 | 2.7141 | 13.1 |
| 33.642 | 2.6619 | 22.4 |
| 34.009 | 2.63402 | 16.7 |
| 36.887 | 2.43478 | 10.2 |
| 37.269 | 2.41075 | 4.8 |
| 37.948 | 2.36913 | 5.1 |
| 38.593 | 2.33102 | 7.3 |
| 39.965 | 2.25409 | 6.5 |
| 40.97 | 2.20108 | 4.6 |
| 41.477 | 2.17535 | 5.8 |
| 43.23 | 2.09112 | 9.4 |
| 45.047 | 2.01092 | 4.3 |
| 45.614 | 1.98722 | 4.9 |

### Example 3

### Preparation of Afatinib Di-Maleate Form E

Afatinib di-maleate form A, prepared according to the procedure disclosed in Example 3 of WO2005/037824 (1 g) was tested for its Hygroscopicity by exposure of form A to different humidity conditions, as presented in the following table.

### Investigation of Hygroscopicity (Dynamic Vapour Sorption (DVS))

### Instrument: SPSx-1μ, (Projekt Messtechnik)

### Temperature: 25° C.

Humidity cycles:

| time [h] | relative humidity [%] |
|---|---|
| 0 | 50 |
| 1.02 | 45 |
| 1.85 | 40 |
| 2.85 | 35 |
| 3.68 | 30 |
| 4.68 | 25 |
| 5.52 | 20 |
| 6.51 | 15 |
| 7.35 | 10 |
| 8.36 | 5 |
| 9.18 | 0 |
| 10.68 | 5 |
| 11.68 | 10 |
| 12.69 | 15 |
| 13.68 | 20 |
| 14.51 | 25 |
| 15.35 | 30 |
| 16.18 | 35 |
| 17.18 | 40 |
| 18.01 | 45 |
| 18.85 | 50 |
| 19.85 | 55 |
| 21.18 | 60 |
| 25.02 | 65 |
| 40.18 | 70 |
| 46.85 | 75 |
| 62.35 | 80 |
| 73.85 | 85 |
| 81.35 | 90 |
| 86.18 | 95 |
| 89.01 | 90 |
| 90.18 | 85 |
| 91.19 | 80 |
| 92.51 | 75 |
| 93.52 | 70 |
| 94.68 | 65 |
| 95.85 | 60 |
| 97.35 | 55 |
| 100.36 | 50 |

| | Afatinib di-maleate | | |
|---|---|---|---|
| | after DVS of Form A | | |
| weight (mg) | 47.28 | 47.45 | 46.06 |
| consumption (mL) | 0.62 | 0.64 | 0.60 |
| water content (% by | 6.95 | 7.15 | 6.90 |
| weight) | | | |

| | |
|---|---|
| average | 7.00 |

The obtained product was analysed by ¹H-NMR Spectroscopy and also by HPLC to confirm there was no decomposition. Then the sample was analyzed by XRD diffraction. The solid state identity is provided in the Table below.

| Angle (2-Theta °) | d value (Angstrom) | Intensity % |
|---|---|---|
| 5.515 | 16.01245 | 34.1 |
| 6.138 | 14.38804 | 12.6 |
| 11.384 | 7.76668 | 18.6 |
| 13.108 | 6.74852 | 14.1 |
| 15.05 | 5.88198 | 14.4 |
| 17.66 | 5.0182 | 22.7 |
| 20.323 | 4.36628 | 27.5 |
| 22.321 | 3.97968 | 35.2 |
| 25.551 | 3.4835 | 100 |
| 28.046 | 3.17897 | 41.3 |
| 29.107 | 3.06542 | 37.5 |

1-11. (canceled)

12. Crystalline Afatinib di-maleate Form D, characterized by data selected from: an X-ray powder diffraction pattern having peaks at 5.6, 9.5, 22.1, 26.3 and 29.5 degrees two theta±0.2 degrees two theta; an X-ray powder diffraction pattern substantially as depicted in FIG. 4; and combinations thereof.

13. The crystalline Afatinib di-maleate Form D of claim 12, characterized by an X-ray powder diffraction pattern having peaks at 5.6, 9.5, 22.1, 26.3 and 29.5 degrees two theta±0.2 degrees two theta.

14. The crystalline Afatinib di-maleate Form D of claim 13, further characterized by additional analytical data selected from: an X-ray powder diffraction pattern having any one, two, three, four, five, six, seven, eight, nine or ten additional peaks selected from peaks at 11.2, 14.4, 18.5, 19.7, 20.5, 20.7, 22.3, 23.5, 24.8 and 28.1 degrees two theta±0.2 degrees two theta; a DSC thermogram substantially as depicted in FIG. 5; and combinations thereof.

15. The crystalline Afatinib di-maleate Form D of claim 12, wherein said form is anhydrous.

16. (canceled)

17. A pharmaceutical composition comprising any one or a combination of Afatinib di-maleate crystalline forms according to claim 12 and at least one pharmaceutically acceptable excipient.

18. (canceled)

19. A method of treating cancer, comprising administering a therapeutically effective amount of at least one Afatinib di-maleate crystalline forms according to claim 12 to a person suffering from cancer.

20. A process for preparing Afatinib free base, or a salt of Afatinib other than a di-maleate salt, or any solid state form of said salt of Afatinib, the process comprising:
a) preparing a crystalline form of Afatinib di-maleate of claim 12;
b) basifying said form to obtain Afatinib free base; and optionally
c) converting the obtained Afatinib free base to a salt of Afatinib other than a di-maleate salt, or a solid state form thereof.

21. The process of claim 20, wherein the conversion of step c) comprises: reacting the obtained Afatinib free base with an appropriate acid to obtain the corresponding acid addition salt.

22. A process for preparing a salt of Afatinib other than a di-maleate salt, or any solid state form of said salt of Afatinib, the process comprising:
a) preparing a crystalline form of Afatinib di-maleate of claim 12; and
b) reacting said crystalline form with an acid having a pKa that is lower than of maleic acid to obtain the corresponding acid addition salt, or a solid state form thereof.
